(19) United States
(12) Patent Application Publication
(10) Pub. No.: US 2003/0059846 A1
(43) Pub. Date: Mar. 27, 2003

(54) Drug interaction assay chip

(76) Inventors: Scott L. Diamond, Bala Cynwyd, PA (US); Christophe J-P. Sevrain, Chicago, IL (US)

Correspondence Address: Barbara E. Johnson, WEBB ZIESENHEIM LOGSDON ORKIN & HANSON, P.C., 700 Koppers Building, 436 Seventh Avenue, Pittsburgh, PA, 15219-1818, US

(21) Appl. No.: 10/224,529

(22) Filed: Aug. 19, 2002

Related U.S. Application Data
(60) Provisional application No. 60/313,366, filed on Aug. 17, 2001.

(51) Int. Cl. G01N 33/53; G01N 33/543

(52) U.S. Cl. 435/7.1; 436/518

(57) ABSTRACT

A tool (a method, and a microarray apparatus) to enable evaluation of drug-drug interactions, by providing large numbers of pharmaceutical compounds on solid substrates in numerous replicate sets suitable for long-term storage. Ordinarily, the various compounds are present in extremely high densities. The libraries of pharmaceutical compounds, when used as a bioreaction assay chip, can be combined with a detection system that includes cells, cellular fractions, enzymes, organic molecules, and fluorescence or chromogenic reporter molecules along with a test drug agent. This method allows the detection of biochemical or biological interaction of the test drug agent with known pharmaceutical compounds in a defined biochemical or biological context of the assay.

### CROSS REFERENCE TO RELATED APPLICATION

This patent application claims priority to U.S. Provisional Application Ser. No. 60/313,366, filed Aug. 17, 2001, which is incorporated herein by reference.

### BACKGROUND OF THE INVENTION

1. Field of the Invention

The invention addresses screening assays for drug/drug interactions, in the pharmaceutical industry.

2. Description of Background

The interaction of one drug with another can present dangerous side effects that are often unknown until they appear in patients. These interactions can be cell or organ specific or involve organ-organ interactions. The interactions may present in only a subset of patients that have a genetic or epigenetic predisposition to side effects due to the drug-drug interaction. A combinatorial assay problem exists where a pharmacological agent of interest (the test compound) is desired to be screened against 10⁵ to 10⁶ known pharmaceutical agents in 10¹ to 10³ of different assays of drug-drug interaction.

Common drug-drug interactions include the effect of antimicrobial agents on the actions of other agents. For example, aminoglycosides can lead to the inactivation of penicillins and/or nephrotoxicity and/or ototoxicity. Nafcillin can reduce the anticoagulant effect of coumarin. Rifampin can reduce the action of oral contraceptives by increased degradation. The need for a tool to evaluate drug-drug interactions acknowledges that many dangerous combinations already exist, but have yet to be identified for better wellness and therapeutic care of animals and patients.

### SUMMARY OF THE INVENTION

The present invention is a tool (a method and a microarray apparatus) to enable evaluation of drug-drug interactions, by providing large numbers of pharmaceutical compounds on glass slides in numerous replicate sets suitable for long-term storage. Ordinarily, the various compounds are present in extremely high densities. The libraries of pharmaceutical compounds, when used as a bioreaction assay chip, can be combined with a detection system that includes cells, cellular fractions, enzymes, organic molecules, and fluorescence or chromogenic reporter molecules along with a test drug agent. This method allows the detection of biochemical or biological interaction of the test drug agent with known pharmaceutical compounds in a defined biochemical or biological context of the assay.

### DESCRIPTION OF THE PREFERRED EMBODIMENT(S)

As described above, the present invention is a tool (a method) to enable evaluation of drug-drug interactions by providing large numbers of pharmaceutical compounds on glass slides in numerous replicate sets suitable for long-term storage. Ordinarily, the various pharmaceutical compounds of interest are present in extremely high densities. The libraries of pharmaceutical compounds, when used as a bioreaction assay chip, can be combined with a detection system that includes cells, cellular fractions, enzymes, organic molecules, and fluorescence or chromogenic reporter molecules along with a test drug agent. This method allows the detection of biochemical or biological interaction of the test drug agent with known pharmaceutical compounds in a defined biochemical or biological context of the assay.

A master library of individual compounds, mixtures of compounds, or reaction premixtures, in solvent, are prepared for storage and subsequent utilization in a Distribution-Ready Library by delivery of the master stock constituents into a distribution formulation liquid (DFL). The individual compounds, within the master library, are generally pharmaceutical compounds of interest, including but by no means limited to, acetaminophen; acetazolamide; ampicillin; aspirin; chlorothiazide; chloropropamide; cromolyn; ethacrynic acid; furosemide; ibuprofen; levodopa; methotrexate; methyldopa; penicillamine; pentobarbital; phenobarbital; phenytoin; propylthiouracil; salicylic acid; sulfadiazine; thyophylline; tolbutamide; warfarin; allopurinol; alprenolol; amiloride; amphetamine; atropine; bupivacaine; chlordiazepoxide; chloroquine; chlorpheniramine; clonidine; cocaine; codeine; cyclizine; desipramine; diazepam; dihydocodeine; diphenhydramine; diphenoxylate; ephedrine; epinephrine; ergotamine; fluphenazine; guanethidine; hydralazine; imipramine; isoproterenol; kanamycin; lidocaine; metraminol; methadone; methamphetamine; methyldopa; methysergide; metoprolol; morphine; nicotine; norepinephrine; pentazocie; phenylephrine; physostigmine; pilocarpine; pindolol; procaine; proazine; proethazine; propranolol; pseudoephrine; pseudoephedrine; pyrimethamine; quinidine; scopolamine; terbutaline; thioridazine; toazoline; taxol; dexamethazone; estrogen; testosterone; estradiol; bumetanide; insulin; tetracycline; cyclosporin; oxytocin; vasopressin; mefenamic acid; piperazine; nystatin; and sodium nitroprusside.

In other words, the pharmaceutical compounds may be selected from the following drug categories, without limitation: acetylcholine receptor stimulants and antagonists; adrenoreceptor-activated drugs; adrenoreceptor-blocking drugs; antihypertensive agents; vasodilators; cardiac glycosides; diuretics; histamine; serotonin; antihistamines; polypeptides; antibiotics; steroids; sedatives; antiepileptic drugs; anesthetics; skeletal muscle relaxants; antidepressants; antipsychotics; analgesics; lithium; anticoagulants; procoagulants; statins; nonsteroidal anti-inflammatory agents; antimitotic agents; protease inhibitors; thyroid and antithyroid drugs; fibrinolytic agents; recombinant proteins; peptides; adrenocorticosteroids; gonadal hormones and inhibitors; penicillins; cephalosporins; chloramphenicol, tetracyclines; polymyxins; antimyobacterial drugs; sulfonamides; trimethoprim; antifingal agents; antiviral agents; anticancer agents; vaccines; antiprotozoal drugs; and antihelminthic drugs.

The Distribution-Ready Library can be maintained indefinitely in storage by virtue of the characteristics of the DFL. At the time of manufacture or time of need, the Distribution-Ready Library is microarrayed onto substrates at high density, thereby creating numerous Library Microarrays that are identical replicates of the master library compound(s) in DFL at fixed and known positions on the substrate. The DFL has a defined surface tension to maintain the master library compound in a non-spreading, non-beading adherent spot at a fixed position on the substrate in a manner that is stable for extended periods of time. The DFL may contain a volatile component that evaporates after microarraying so as to reduce the adherent spot size. Chemical linkage of the pharmaceutical compound(s) to the slide is not required.

An important feature of the invention is the DFL, which has a defined composition to maintain the constituents of the master library in a stable form for long-term storage. The DFL has a defined composition so as to display a surface tension to maintain the master library compound in a non-spreading, non-beading adherent droplet at a fixed position on a particular substrate of choice in a manner that is stable for extended periods of time after arraying. DFL is usually, if not always: miscible with water; miscible with common organic solvents such as DMSO, ethanol, methanol, etc.; moderately viscous, with a viscosity between 1-10,000 Centipoise; compatible with biological molecules and biological reagents such as nucleic acid, peptides, proteins, sugars, or small 20 nanometer to 200 nanometer microcarrier beads; adequately fluid for movement into and out of microcapillary devices such as hollow tips, microarray pins, or microsyringes used for arraying; able to create a specific contact angle to form a stable finite lens where the bioreaction fluid in the spot after arraying does not spread (contact angle >0) but wherein the stable adherent lens thus formed does not have too low of adhesion that the spot can roll on the substrate (contact angle <90); and low enough in volatility of one component such that the reaction zone does not completely evaporate. The DFL may contain a volatile component (the volatile solvent) and a non-volatile component (termed the carrier solvent) that is suitable for applying small volumes of a fluid mixture to a surface by microarraying or positive displacement whereby evaporation of the volatile solvent results in highly localized, long-lasting liquid microdot (or spot) residues of master mixture components. The carrier solvent may be such that the volatile solvent in the DFL is suitable for obtaining a true solution of fluorogenic or chromogenic substrates at high concentration. This solvent may be DMSO (dimethylsulfoxide); chloroform; acetone; 5% acetic acid; water; an alcohol such as methanol, ethanol or propanol; ethyl ether; or alkane.

Using the above-described DFL, the Distribution-Ready Library is constructed with the requirement of preserving the library indefinitely in storage and maintaining a suitable environment for subsequent microarraying manipulations. The individual members of the master library are added to wells that are preloaded with the DFL. For example, a fixed volume of liquid (1 to 50 microliters) may be removed from the master library well and charged to a multi-well plate well containing 10 to 200 microliters of the DFL to yield the Distribution-Ready Library. The Distribution-Ready Library can be utilized for microarraying, stored at room temperature or at refrigerated temperatures (4° C.) or at frozen temperatures (0° C., −20° C., or −80° C.). The Distribution-Ready Library can likewise be maintained in multi-well plates, including but not limited to, 96-well, 384-well and 1536-well plates. Due to the composition of the DFL, the Distribution-Ready Library is well-suited for long-term storage and stability under any of the above circumstances.

Additional features of the DFL are as follows. The DFL may contain a carrier solvent which is of low volatility, miscible with any volatile solvent, or miscible with water containing biological fluids. The DFL is in many cases suitable for maintaining a true solution of fluorogenic or chromogenic substrate at high concentration after evaporation of the volatile solvent. The carrier solvent may be a polyalcohol such as 1,2-ethanediol; 2,3-butanediol; or 1,2,3-propanetriol (glycerol). The carrier solvent of the DFL may contain viscosity enhancers such as dextran, pluronic acid, carbohydrates of the pentose, ribose or hexose families and related polysaccharides, or polyethylene glycol polymers. The carrier solvent may include fluorogenic substrates, chromogenic substrates, enzyme co-factors, inhibitors, or activators. Volatile solvent facilitates fluid handling and delivery by reduction of formulation viscosity. Evaporation of the volatile solvent facilitates additional concentrating of non-volatile reactive components. The non-volatile carrier solvent and its constituents represent a high viscosity fluid with significant yield stress and surface tension to resist fluid motion. The non-volatile carrier solvent and its constituents allow for the maintaining of the fluorogenic or chromogenic substrate and co-factors and inhibitors or activators or other biological additives to remain in the liquid state without crystallization or precipitation. The DFL may contain buffering agents, chelating agents, an antioxidant, a reducing agent such as beta-mercaptoethanol, or antimicrobial agents as preservatives.

Sample formulas for the DFL are provided as follows, with any of the following being very typical DFL formulations: 50% glycerol, 10% DMSO, and 40% water; 80% glycerol, 10% DMSO, and 10% water; 50% ethylene glycol, 10% DMSO, and 40% water; 90% glycerol, and 10% water; and 90% glycerol and 10% DMSO.

At the time of manufacture or at the time of need, the Distribution-Ready Library is microarrayed onto substrates at high density, thereby creating numerous Library Microarrays or individual Microarrayed Library sets that are identical replicates of the master library compound(s) in DFL and are resident in the DFL at fixed and known positions on the substrate. The volatile constituents of the DFL can evaporate rapidly due to the high surface area to volume ratio of each spot on the microarray. Microarraying is achieved by solid or hollow tip pins, positive displacement through micro-needles, or ink-jet printing methods. For example, 10 plates of a Distribution-Ready Library of 384 wells can be arrayed onto 50 or 100 slides, where each slide contains 10×384-3840 pharmaceutical compounds resident in the DFL at known positions on the slide. Chemical linkage of the compound to the slide is not required. The Library Microarrays are suitable for the conducting of chemical and biochemical reactions, exposure to electromagnetic radiation, or exposure to living cells or cell fractions.

Whereas the master compound in the master library is maintained at a 10×to 1000×concentration of constituents in preparation for defined dilution events where the master mixture is ultimately diluted to a 1×concentration at time of final utilization. For example, a master well can contain a 1 millimolar concentration of dexamethasone in DMSO that is diluted 10000×to 0.1 micromolar upon formation of the Distribution-Ready Library and diluted further 2×upon usage in a final assay reaction where the desired final concentration of the dexamethasone is 50 nanomolar.

The final volume of the spots on the microarray, after evaporation of the volatile solvent, can range from about 1 to about 50 nanoliters. Separation between microdot edges is set at about 10 to about 1000 micrometers. Surfaces for delivery of liquid from the Distribution-Ready Library include silicon, glass, silica, quartz, polystyrene, nylon membranes, or other porous or non-porous polymeric membranes. When samples of 50 or 100 replicates are microarrayed from the Distribution-Ready Library, each well can be sampled at least 1000 times per 100 microliters of volume of the Distribution-Ready Library.

Microarrays prepared, as discussed above, may be used to assay one or more test compounds for determination of drug-drug interactions. Such assays may be conducted using fluorogenic detection systems for activation of an enzyme, inhibition of an enzyme pathway, production of reaction product, or change in cell biology property such as membrane permeability, ionic conductance, ion transport, or ion channel regulation. The test agent can be evaluated as to all the different compounds on the microarray for binding, creation of new compounds, reaction kinetics or any other discernible result of the admixture of the test substance with the compounds on the microarray. Because the master library is created in the first place in such a way as to keep all elements of the library separate, usually in storage in multi-well plates, only a single compound is present in any given spot on the microarray, allowing for differential assays to be performed simultaneously for scores if not hundreds of drugs.

Exemplary applications of the invention include treating a microarrayed drug library with a microsomal preparation of liver enriched in metabolic enzymes such as the P450 pathway followed by delivery of the test compound. The presence of degradation of the test compound can be discerned by a competition assay of the unknown level of the test compound with a known level of the fluorogenic substrate and its converting enzyme. Alternatively, the present microarrays may be used for urine or other body fluid assays for detection of precipitates, or with test compound(s) and living cells or any other biological materials for drug-drug interaction assays.

The invention claimed is:

1. A method for creating a storable distribution-ready library for evaluating drug-drug interactions, comprising formulating a distribution formulation liquid and admixing individual aliquots of distribution formulation liquid with individual samples of pharmaceutical compounds from a master library, wherein said storable distribution-ready library thus formed is characterized by its ability to form microarrays in which the spots formed therefrom are non-spreading, non-beading, and stable for an extended period of time.

2. The method according to claim 1, wherein the spots of the microarray individually contain a compound selected from the group consisting of acetaminophen; acetazolamide; ampicillin; aspirin; chlorothiazide; chloropropamide; cromolyn; ethacrynic acid; furosemide; ibuprofen; levodopa; methotrexate; methyldopa; penicillamine; pentobarbital; phenobarbital; phenytoin; propylthiouracil; salicylic acid; sulfadiazine; thyophylline; tolbutamide; warfarin; allopurinol; alprenolol; amiloride; amphetamine; atropine; bupivacaine; chlordiazepoxide; chloroquine; chlorpheniramine; clonidine; cocaine; codeine; cyclizine; desipramine; diazepam; dihydrocodeine; diphenhydramine; diphenoxylate; ephedrine; epinephrine; ergotamine; fluphenazine; guanethidine; hydralazine; imipramine; isoproterenol; kanamycin; lidocaine; metraminol; methadone; methamphetamine; methyldopa; methysergide; metoprolol; morphine; nicotine; norepinephrine; pentazocie; phenylephrine; physostigmine; pilocarpine; pindolol; procaine; proazine; proethazine; propranolol; pseudoephrine; pseudoephedrine; pyrimethamine; quinidine; scopolamine; terbutaline; thioridazine; toazoline; taxol; dexamethazone; estrogen; testosterone; estradiol; bumetanide; insulin; tetracycline; cyclosporin; oxytocin; vasopressin; mefenamic acid; piperazine; nystatin; and sodium nitroprusside.

3. The method according to claim 2, wherein the master library contains compounds from categories selected from the group consisting of acetylcholine receptor stimulants and antagonists; adrenoreceptor-activated drugs; adrenoreceptor-blocking drugs; antihypertensive agents; vasodilators; cardiac glycosides; diuretics; histamine; serotonin; antihistamines; polypeptides; antibiotics; steroids; sedatives; antiepileptic drugs; anesthetics; skeletal muscle relaxants; antidepressants; antipsychotics; analgesics; lithium; anticoagulants; procoagulants; statins; nonsteroidal anti-inflammatory agents; antimitotic agents; protease inhibitors; thyroid and antithyroid drugs; fibrinolytic agents; recombinant proteins; peptides; adrenocorticosteroids; gonadal hormones and inhibitors; penicillins; cephalosporins; chloramphenicol, tetracyclines; polymyxins; antimyobacterial drugs; sulfonamides; trimethoprim; antifungal agents; antiviral agents; anticancer agents; vaccines; antiprotozoal drugs; and antihelminthic drugs.

4. The method according to claim 2, wherein the distribution formulation liquid has a defined surface tension to maintain the master library compound in a non-spreading, non-beading adherent spot at a fixed position on the microarray in a manner that is stable for extended periods of time.

5. The method according to claim 4, wherein the distribution formulation liquid contains at least one constituent selected from the group consisting of glycerol, ethylene glycol, dimethylsulfoxide, and water.

6. The method according to claim 4, wherein the distribution formulation liquid contains at least one volatile component.

7. The method according to claim 6, wherein the distribution formulation liquid further is miscible with water and has a viscosity between 1-10,000 Centipoise.

8. The method according to claim 7, wherein the distribution formulation liquid further contains at least one compound selected from the group consisting of polyalcohol, viscosity enhancer, saccharide, and polyalkylene glycol polymer.

9. A microarray comprising a solid substrate, and a plurality of spots on said substrate, with each spot comprising an admixture of a pharmaceutical compound from a master library and a distribution formulation liquid, wherein the distribution formulation liquid has a surface tension adequate to maintain the spots in a non-spreading, non-beading configuration, and further wherein no chemical constituent of the master library is covalently linked or otherwise chemically bonded to the substrate except by the adherence created by the distribution formulation liquid.

10. The microarray according to claim 9, wherein said distribution formulation liquid contains at least one compound selected from the group consisting of glycerol, ethylene glycol, dimethylsulfoxide, and water.
